(19) United States
(12) Patent Application Publication
(10) Pub. No.: US 2003/0168357 A1
(43) Pub. Date: Sep. 11, 2003

(54) Disposable teethwipe packet

(76) Inventors: Annie Lois Campbell, St. Petersburg, FL (US); Will Leonard Nelson, St. Petersburg, FL (US)

Correspondence Address: Annie L. Campbell, 2133 22nd Avenue S., St. Petersburg, FL, 33712, US

(21) Appl. No.: 10/078,761

(22) Filed: Feb. 19, 2002

(51) Int. Cl. A61B 19/02

(52) U.S. Cl. 206/63.5

(57) ABSTRACT

The present invention provides a pre-moistened wet wipe comprising of one of the following: mouthwash; toothpaste; or breath freshener, in a single packet.

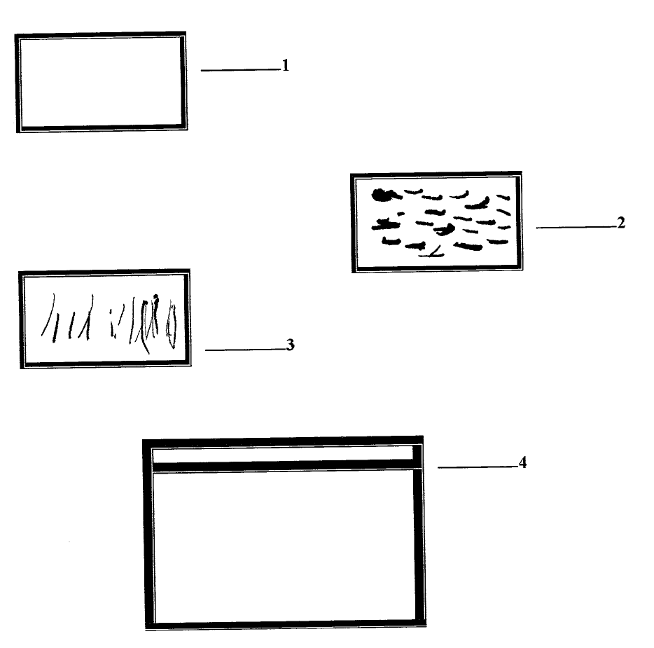

### CROSS-REFERENCE TO RELATED APPLICATION

CROSS-REFERENCE TO RELATED APPLICATION

| | | | |
|---|---|---|---|
| 4,071,300 | January, 1978 | Nichols, et al. | 728/435 |
| 4,176,980 | Decmeber, 1979 | O'Neal, et al. | 857/008 |
| 4,221,492 | September, 1980 | Boscardin, et al. | 856/360 |
| 5,575,228 | November, 1996 | Zimmerman, et al. | 552/692 |
| 5,582,195 | December, 1996 | Nagel | 329/265 |
| 5,629,081 | May, 1997 | Richards et al. | 414/540 |
| 5,660,546 | August, 1997 | Shafer | 531/725 |
| 5,575,228 | November, 1996 | Padgett, III et al. | 112/662 |
| 5,713,492 | February, 1998 | DeGennaro | 558/836 |
| 5,881,421 | March, 1999 | Ducharme | 822/619 |
| 6,077,501 | June, 2002 | Sickora, et al. | 107/312 |
| 6,258,057 B1 | July, 2001 | Marinello et al. | 09/271/354 |
| 6,280,529 B1 | August, 2001 | Meyer | 09/248/368 |
| 6,341,688 | January, 2002 | Graham | 687/919 |
| D,427,686 | July, 2000 | Butler, et al. | 086/765 |

### BACKGROUND OF THE INVENTION

Personal cosmetic dental hygiene consist of regular brushing with toothpaste, flossing, and rinses in a effort of reducing gum diseases and/or cavities; Pre-moistened wipes, prepared from paper products are used as wash cloths in cleansing the skin when soap and water is not available to clean the skin of the body disposable toothbrushes with toothpaste are available for brushing the teeth. Although, there are many disposable wetwipes consisting of soaps and lotions for cleansing and refreshing the skin, and many disposable toothbrushes consisting of dental cleansers for cleansing the teeth; there is no disposable wetwipe consisting of teeth or denture cleansing and refreshing products for when teeth and denture cleaning tools are not available, or when dental problems won't permit brushing. Many people have had an tooth abscess, and during this time using a toothbrush may cause pain. With this invention you can wipe your teeth clean until your gum heals to resume using a toothbrush.

### BRIEF SUMMARY OF THE INVENTION

This invention relates to the wiping of teeth when a abscess, swelling, and/or soreness of gums makes brushing with a toothbrush unbearable; After brushing with a toothbrush, the teeth may need wiping instead of brushing, or dentures may need wiping instead of soaking, sometimes due to debris from eating or cosmetics that was applied to the lips gets on the teeth. The present invention is a refresher teeth or denture wipe to be used when unable to brush teeth, or soak denture, after eating in a restaurant you may need to fresh up your breath, teeth, or denture in the privacy of your car, office, etc., before pursuing on to other activities. Brushing the teeth several times a day, or soaking your dentures is an activity most of us do not have time for, and too much brushing of your teeth, or soaking of your dentures may cause irritation to the gum; so in between brushing our teeth, or soaking of dentures, wiping the teeth or dentures several times a day will not cause gum irritation.

### BRIEF DESCRIPTION OF THE SEVERAL VIEWS OF THE DRAWING

The Drawings shows a view of a wetwipe consisting of toothpaste (1); A view of a wetwipe consisting of mouthwash (2); View of a wetwipe consisting of breathfreshener (3); for sanitary storage the disposable teethwipe in package (4).

### DETAILED DESCRIPTION OF THE INVENTION

A sterilized paper product wetwipe is diluted in various mint or non-mint flavors of toothpaste. Then placed in a sealed sterilized single or multiple package, labeled “Toothpaste Teethwipe” or “Toothpaste Teethwipes”.

A sterilized paper product wetwipe is diluted in various mint or non-mint flavors of mouthwash. Then placed in a sealed sterilized single or multiple package, labeled “Mouthwash Teethwipe” or “Mouthwash Teethwipes”.

A sterilized paper product wetwipe is diluted in various mint or non-mint flavors of breath freshener. Then placed in a sealed sterilized single or multiple package, labeled “Breath Freshener Teethwipe” or “Breath Freshener Teethwipes”.

Directions on how to use: Tear Teethwipe package to remove wetwipe. Rub back and forth across the teeth or dentures in front and behind. Throw away used wetwipe.

What I claim as my invention is:

1. Accordingly stated, the disposable teethwipe packet is the basis of the present invention.

2. Wetwipes comprising of toothpaste to be used to wipe the teeth.

3. Wetwipes comprising of mouthwash to be used to wipe the teeth.

4. Wetwipes comprising of breath fresher to be used to wipe the teeth.
